(19) United States
(12) Patent Application Publication
(10) Pub. No.: US 2016/0152709 A1
(43) Pub. Date: Jun. 2, 2016

(54) COMPOSITIONS AND METHODS FOR TREATMENT OF STROKE

(71) Applicant: Biogen MA Inc., Cambridge, MA (US)

(72) Inventors: Barry Ticho, Newton, MA (US); Jacob Elkins, Brookline, MA (US)

(21) Appl. No.: 14/902,911

(22) PCT Filed: Jul. 3, 2014
(86) PCT No.: PCT/US14/45457

§ 371 Date: Jan. 5, 2016

Related U.S. Application Data
(60) Provisional application No. 61/843,125, filed on Jul. 5, 2013.

(51) Int. Cl. C07K 16/28; A61K 45/06; A61K 39/395

(52) U.S. Cl. CPC C07K 16/2842; A61K 39/3955; A61K 45/06; C07K 2317/21; C07K 2317/24; C07K 2317/76; C07K 2317/55; C07K 2317/54; A61K 2039/505

(57) ABSTRACT

The invention relates, inter alia, to methods of treating stroke, e.g., ischemic stroke, e.g., acute ischemic stroke, and methods of reducing infarct size and/or other neurological deficits associated with stroke, e.g., ischemic stroke, e.g., acute ischemic stroke, using a VLA-4 antagonist such as natalizumab. It was discovered that VLA-4 antagonists such as natalizumab can effectively reduce the infarct size and other associated neurological deficits of a stroke, e.g., an ischemic stroke; e.g., an acute ischemic stroke, e.g., when administered within a specified time period after the onset of the stroke. Also disclosed herein are articles of manufacture and kits for the treatment of stroke, e.g., ischemic stroke, e.g., acute ischemic stroke.

### CROSS-REFERENCE TO RELATED APPLICATIONS

This application claims the benefit of U.S. Provisional Application No. 61/843,125, filed Jul. 5, 2013, the entire contents of which are incorporated herein by reference.

### FIELD OF THE INVENTION

The invention relates to compositions and methods for treating stroke and/or other neurological deficits associated with stroke.

### BACKGROUND OF INVENTION

Stroke occurs when there is an interruption of blood flow to the brain, causing the death of neuronal tissue and focal neurological deficits. The signs and symptoms may vary with the location and extent of the stroke. There are nearly 800,000 strokes of all types per year in the United States, and ischemic strokes account for approximately 80% of these strokes. Roger et al. (2011) Circulation 123(4):e18-e209. In Europe, the estimated annual incident of stroke is over 1.1 million, with a similar percentage of these, approximately 80%, being ischemic strokes. Heuschmann et al. (2009) Stroke 40(5):1557-1563.

Guidelines for the evaluation and treatment of acute stroke patients focus on reperfusion therapies and factors that may exacerbate stroke or complicate clinical course. The diagnosis of acute ischemic stroke is made through a combination of a history and physical examination that is consistent with focal ischemia and a resulting neurological deficit. Brain imaging, either computed tomography (CT) or magnetic resonance imaging (MRI) is used to exclude hemorrhage and other focal pathologies and document early signs of ischemia.

Recombinant tissue plasminogen activator (rtPA) is the only approved pharmacological therapy for acute ischemic stroke. It is approved for use within 3 hours of stroke onset in the United States and within 4.5 hours in many European countries. Current American Heart Association guidelines also suggest use up to 4.5 hours after stroke onset, although treatment effects diminish over time and risk of hemorrhage increases (Jauch (2013)). Because of the narrow time window, it is estimated that only 3% of patients with stroke receive rtPA.

### SUMMARY OF INVENTION

The invention relates, inter alia, to methods of treating stroke, e.g., ischemic stroke, e.g., acute ischemic stroke, and methods of reducing infarct size and/or other neurological deficits associated with stroke, e.g., ischemic stroke, e.g., acute ischemic stroke, using a VLA-4 antagonist such as natalizumab. It was discovered that VLA-4 antagonists such as natalizumab can effectively reduce the infarct size and other associated neurological deficits of a stroke, e.g., an ischemic stroke, e.g., an acute ischemic stroke, e.g., when administered within a specified time period after the onset of the stroke. For example, the administration of a VLA-4 antagonist such as natalizumab to a subject within a period of nine hours or less, e.g., 8, 7, 6 hours or less, after the onset of a stroke, e.g., an ischemic stroke, provides an effective treatment against the secondary injuries associated with stroke. Also disclosed herein are articles of manufacture and kits for the treatment of stroke, e.g., ischemic stroke, e.g., acute ischemic stroke.

Accordingly, in one aspect, the disclosure features a method of treating a human subject having a stroke, e.g., an ischemic stroke, e.g., an acute ischemic stroke, comprising: administering a VLA-4 antagonist to the subject within 12 hours or less, e.g., 10, 9, 8, 7, 6 hours or less, after the onset of the stroke in the subject. In some embodiments, the VLA-4 antagonist is administered within 9 hours or less after the onset of the stroke, e.g., between 6 and 9 hours after the onset of the stroke. In particular embodiments, the VLA-4 antagonist is administered within 6 hours or less after the onset of the stroke, e.g., between 3 and 6 hours, 4.5 to 6 hours, 5 to 6 hours, after the onset of the stroke.

In another aspect, the disclosure features a method of treating a subject, e.g., human subject, having a stroke, e.g., an ischemic stroke, e.g., an acute ischemic stroke, comprising: administering a VLA-4 antagonist to the subject within more than 2 hours to 12 hours, e.g., more than 2 hours to 10 hours or less, more than 2 hours to 9 hours or less, more than 2 hours to 8 hours or less, more than 2 hours to 7 hours or less, more than 2 hours to 6 hours or less, after the onset of stroke in the subject. In some embodiments, the VLA-4 antagonist is administered within more than 2 to 9 hours or less after the onset of the stroke, e.g., between 6 and 9 hours after the onset of the stroke. In particular embodiments, the VLA-4 antagonist is administered within more than 2 hours to 6 hours or less after the onset of the stroke, e.g., between 3 and 6 hours, 4.5 to 6 hours, 5 to 6 hours, after the onset of the stroke.

In certain embodiments, the a4 antagonist is an anti-VLA-4 antibody molecule, e.g., an anti-VLA-4 antibody molecule described herein. In particular embodiments, the anti- VLA-4 antibody molecule is a monoclonal, a humanized, a human, a chimeric anti-VLA-4 antibody molecule. In some embodiments, the VLA-4 antagonist is an α4-binding fragment of an anti-VLA-4 antibody. In certain embodiments, the α4 binding fragment is an Fab, Fab′, F(ab′)₂, or Fv fragment.

In some embodiments, the anti- VLA-4 antibody molecule comprises one or more, preferably all, of HC CDR1, HC CDR2, HC CDR3, LC CDR1, LC CDR2 and LC CDR3 of natalizumab.

In certain embodiments, the a4 antagonist is natalizumab. In some embodiments, natalizumab is administered at a dose of 200 mg to 400 mg, e.g., 250 mg to 350 mg, e.g., 300 mg, e.g., by intravenous administration, e.g., over a period of less than 90 minutes, e.g., 30 to 60 minutes.

In some embodiments, the stroke is a grade 4 stroke or higher as defined by the National Institute of Health Stroke Scale (NIHSS). In some embodiments, the stroke is a grade 6 stroke or lower as defined by the National Institute of Health Stroke Scale (NIHSS), e.g., between a grade 4 and a grade 6 stroke. In certain embodiments, the stroke is a moderate stroke, a moderate to severe stroke or a severe stroke. In particular embodiments, the stroke is a embolism-, thrombus- or hypoperfusion-associated stroke. In certain embodiments, the subject having the stroke does not have an intracranial hemorrhage.

In some embodiments, the subject has not received a previous treatment with a VLA-4 antagonist, e.g., natalizumab. In some embodiments, the subject does not have or is not at risk for developing progressive multifocal leukoencephalopathy (PML).

In certain embodiments, the α4 antagonist is administered in combination with an additional agent or procedure. For example, in some embodiments, the α4 antagonist is administered simultaneously with an additional agent or procedure. In certain embodiments, the α4 antagonist is administered sequentially with an additional agent or procedure. In particular embodiments, the α4 antagonist is administered, e.g., 30 minutes, 1 hour, 2 hours, 3 hours, 4 hours, 5 hours, or more, after the additional agent or procedure. In particular embodiments, the α4 antagonist is administered, e.g., 30 minutes, 1 hour, 2 hours, 3 hours, 4 hours, 5 hours, or more, before the additional agent or procedure.

In some embodiments, the additional agent ameliorates one or more side effected associated with the administration of the VLA-4 antagonist, e.g., an agent which reduces or inhibits one or more symptom of hypersensitivity. For example, in some embodiments, the agent which reduces or inhibits one or more symptoms of hypersensitivity can be one or more of a corticosteroid (e.g., dexamethasone), an antihistamine (e.g., diphenhydramine), an H1 antagonist and an H2 antagonist (e.g., ranitidine or famotidine). In certain embodiments, the additional agent is an agent which reduces one of more symptom of stroke.

In another aspect, the disclosure features a method of treating a human subject having an ischemic stroke, e.g., an acute ischemic stroke, comprising: administering natalizumab to the subject within 9 hours or less, e.g., 8, 7, 6 hours or less, after the onset of the stroke in the subject.

In a further aspect, the disclosure features a method of treating a subject, e.g., human subject, having an ischemic stroke, e.g., an acute ischemic stroke, comprising: administering natalizumab to the subject within more than 2 hours to 9 hours or less, e.g., more than 2 hours to 8 hours or less, more than 2 hours to 7 hours or less, more than 2 hours to 6 hours or less, after the onset of the stroke in the subject.

In some embodiments, the natalizumab is administered at a dose of 300 mg, e.g., by intravenous administration, e.g., over a period of 30 to 60 minutes. In various embodiments, the stroke is an acute ischemic stroke of a grade 4 stroke or higher as defined by the National Institute of Health Stroke Scale (NIHSS). In some embodiments, the stroke is a grade 6 stroke or lower as defined by the National Institute of Health Stroke Scale (NIHSS), e.g., between a grade 4 and a grade 6 stroke. In certain embodiments, the stroke is a moderate stroke, a moderate to severe stroke or a severe stroke. In some embodiments, the stroke is a embolism-, thrombus- or hypoperfusion-associated stroke.

In certain embodiments, the subject has not received a previous treatment with natalizumab. In some embodiments, the subject does not have or is not at risk for developing progressive multifocal leukoencephalopathy (PML).

In another aspect, the disclosure features a method of treating a subject having an acute ischemic stroke of a grade 4 stroke or higher as defined by the National Institute of Health Stroke Scale (NIHSS), comprising: administering a VLA-4 antagonist to the subject after the onset of the stroke in the subject. In some embodiments, the VLA-4 antagonist is an anti-VLA-4 antibody molecule, e.g., described herein. In certain embodiments, the VLA-4 antagonist is administered at a dose and/or dosing schedule described herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description, and from the claims.

### DETAILED DESCRIPTION

The disclosure is based, at least in part, on the discovery that VLA-4 antagonists such as natalizumab can effectively reduce the infarct size and other associated neurological deficits of a stroke, e.g., an ischemic stroke, e.g., an acute ischemic stroke, e.g., when administered within a specified time period after the onset of the stroke. For example, the administration of a VLA-4 antagonist such as natalizumab to a subject within a period of nine hours or less, e.g., 8, 7, 6 hours or less, after the onset of a stroke, e.g., an ischemic stroke, e.g., acute ischemic stroke, provides an effective treatment against the secondary injuries associated with stroke. Thus, treatment of stroke, e.g., acute ischemic stroke, with a VLA-4 antagonist provides an extended time period for treating subjects having a stroke as compared to other approved treatments such as rtPA.

The following definitions are provided for specific terms used in the following written description and appended claims.

The integrin very late antigen (VLA) superfamily is made up of structurally and functionally related glycoproteins consisting of (alpha and beta) heterodimeric, transmembrane receptor molecules found in various combinations on nearly every mammalian cell type. (For reviews see: E. C. Butcher, Cell, 67, 1033 (1991); D. Cox et al., “The Pharmacology of the Integrins.” Medicinal Research Rev. (1994) and V. W. Engleman et al., “Cell Adhesion Integrins as Pharmaceutical Targets” in Ann. Report in Medicinal Chemistry, Vol. 31, J. A. Bristol, Ed.; Acad. Press, NY, 1996, p. 191). Integrins of the VLA family include (at present) VLA-1, -2, -3, -4, -5, -6, -9, and -11 in which each of the molecules comprise a β1 chain non-covalently bound to an α chain, (α1, α2, α3, α4, α5, α6 and the like), respectively.

Alpha 4 beta 1 (α4β1) integrin is a cell-surface receptor for VCAM-1, fibronectin and possibly other ligands (the latter ligands individually and collectively referred to as “alpha4 ligand(s)”). The term α4β31 integrin (“VLA-4” or “a4b1” or “a4b1 integrin”, used interchangeably herein) refers to polypeptides which are capable of binding to VCAM-1 and members of the extracellular matrix proteins, most particularly fibronectin, or fragments thereof, although it will be appreciated by persons of ordinary skill in the art that other ligands for VLA-4 may exist and can be analyzed using conventional methods. Nevertheless, it is known that the alpha4 subunit will associate with other beta subunits besides betal so the term “alpha 4 integrin” or “alpha 4 subunit-containing integrin”, as used herein, refers to those integrins whose α4 subunit associates with one or another of the beta subunits. Another example of an “α4” integrin besides VLA4 is alpha4beta7 (α4β7) (See Lobb and Adams, supra).

Also included in the methods described herein are molecules that antagonize the action of more than one α4 subunit-containing integrin, such as small molecules or antibody molecules that antagonize both VLA-4 and α4β7 or other combinations of α4 subunit-containing integrins.

Also included within the scope are methods using a combination of molecules such that the combination antagonizes the action of more than one integrin, such as methods using several small molecules or antibody molecules that in combination antagonize both VLA-4 and α4β7 or other combinations of α4 subunit-containing integrins.

“Covalently coupled”—means that the specified moieties (e.g., PEGylated VLA-4 antagonist, immunoglobulin fragment/VLA-4 antagonist) are either directly covalently bonded to one another, or else are indirectly covalently joined to one another through an intervening moiety or moieties, such as a spacer moiety or moieties. The intervening moiety or moieties are called a “coupling group”. The term “conjugated” is used interchangeably with “covalently coupled”. In this regard a “spacer” refers to a moiety that may be inserted between an amino acid or other component of a VLA-4 antagonist and the remainder of the molecule. A spacer may provide separation between the amino acid or other component and the rest of the molecule so as to prevent the modification from interfering with protein function and/or make it easier for the amino acid or other component to link with another moiety.

“Expression vector,” as used herein refers to a polynucleotide, such as a DNA plasmid or phage (among other common examples) which allows expression of at least one gene when the expression vector is introduced into a host cell. The vector may, or may not, be able to replicate in a cell.

“Functional equivalent” of an amino acid residue is (i) an amino acid having similar reactive properties as the amino acid residue that was replaced by the functional equivalent; (ii) an amino acid of an antagonist of the invention, the amino acid having similar properties as the amino acid residue that was replaced by the functional equivalent; (iii) a non-amino acid molecule having similar properties as the amino acid residue that was replaced by the functional equivalent.

A first polynucleotide encoding a proteinaceous antagonist of the invention is “functionally equivalent” compared with a second polynucleotide encoding the antagonist protein if it satisfies at least one of the following conditions:
(a): the “functional equivalent” is a first polynucleotide that hybridizes to the second polynucleotide under standard hybridization conditions and/or is degenerate to the first polynucleotide sequence. Most preferably, it encodes a mutant protein having the activity of a VLA-4 antagonist protein;
(b) the “functional equivalent” is a first polynucleotide that codes on expression for an amino acid sequence encoded by the second polynucleotide.

The VLA-4 antagonists include, but are not limited to, the agents listed herein as well as their functional equivalents. As used herein, the term “functional equivalent” therefore refers to a VLA-4 antagonist or a polynucleotide encoding the VLA-4 antagonist that has the same or an improved beneficial effect on the recipient as the VLA-4 antagonist of which it is deemed a functional equivalent. As will be appreciated by one of ordinary skill in the art, a functionally equivalent protein can be produced by recombinant techniques, e.g., by expressing a “functionally equivalent DNA”. Accordingly, the disclosure embraces integrin proteins encoded by naturally-occurring DNAs, as well as by non-naturally-occurring DNAs which encode the same protein as encoded by the naturally-occurring DNA. Due to the degeneracy of the nucleotide coding sequences, other polynucleotides may be used to encode integrin protein. These include all, or portions of the above sequences which are altered by the substitution of different codons that encode the same amino acid residue within the sequence, thus producing a silent change. Such altered sequences are regarded as equivalents of these sequences. For example, Phe (F) is coded for by two codons, TTC or TTT, Tyr (Y) is coded for by TAC or TAT and His (H) is coded for by CAC or CAT. On the other hand, Trp (W) is coded for by a single codon, TGG. Accordingly, it will be appreciated that for a given DNA sequence encoding a particular integrin there will be many DNA degenerate sequences that will code for it. These degenerate DNA sequences are considered within the scope of this disclosure.

The term “chimeric”, when referring to an antagonist, means that the antagonist is comprised of a linkage (chemical cross-linkage or covalent or other type) of two or more proteins having disparate structures and/or having disparate sources of origin. Thus, a chimeric VLA-4 antagonist may include one moiety that is a VLA-4 antagonist or fragment and another moiety that is not a VLA-4 antagonist.

A species of “chimeric” protein is a “fusion” or “fusion protein” which refers to a co-linear, covalent linkage of two or more proteins or fragments thereof via their individual peptide backbones, most preferably through genetic expression of a polynucleotide molecule encoding those proteins. Thus, preferred fusion proteins are chimeric proteins that include a VLA-4 antagonist or fragment covalently linked to a second moiety that is not a VLA-4 antagonist. Preferred fusion proteins include portions of intact antibodies that retain antigen-binding specificity, for example, Fab fragments, Fab′ fragments, F(ab′)2 fragments, F(v) fragments, heavy chain monomers or dimers, light chain monomers or dimers, dimers consisting of one heavy and one light chain, and the like.

The other preferred fusion proteins are chimeric and comprise a VLA-4 antagonist moiety fused or otherwise linked to all or part of the hinge and constant regions of an immunoglobulin light chain, heavy chain, or both. Thus, the methods described herein can utilize a molecule that include: (1) an VLA-4 antagonist moiety, (2) a second peptide, e.g., one which increases solubility or in vivo life time of the VLA-4 antagonist moiety, e.g., a member of the immunoglobulin super family or fragment or portion thereof, e.g., a portion or a fragment of IgG, e.g., the human IgG1 heavy chain constant region, e.g., CH2, CH3, and hinge regions. Specifically, a “VLA-4 antagonist/lg fusion” is a protein comprising a biologically active VLA-4 antagonist (e.g. a soluble VLA-4 ligand), or a biologically active fragment thereof linked to an N-terminus of an immunoglobulin chain wherein a portion of the N-terminus of the immunoglobulin is replaced with the VLA-4 antagonist. A species of VLA-4 antagonist/lg fusion is a “VLA-4/Fc fusion” which is a protein comprising a VLA-4 antagonist, e.g., described herein, linked to at least a part of the constant domain of an immunoglobulin. A preferred Fc fusion comprises a VLA-4 antagonist, e.g., described herein, linked to a fragment of an antibody containing the C terminal domain of the heavy immunoglobulin chains.

The term “fusion protein” also means a VLA-4 antagonist chemically linked via a mono- or hetero-functional molecule to a second moiety that is not a VLA-4 antagonist (resulting in a “chimeric” molecule). Thus, one example of a chemically linked, as opposed to recombinantly linked, chimeric molecule that is a fusion protein may comprise: (1) VLA-4 subunit targeting moiety, e.g., a VCAM-1 moiety capable of binding to VLA-4) on the surface of VLA-4 bearing cells; (2) a second molecule which increases solubility or in vivo life time of the targeting moiety, e.g., a polyalkylene glycol polymer such as polyethylene glycol (PEG). The VLA-4 targeting moiety can be any naturally occurring VLA-4 ligand or fragment thereof, e.g., a VCAM-1 peptide or a similar conservatively substituted amino acid sequence.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences.

As used herein, the term “hybridizes under high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. High stringency hybridization conditions include hybridization in 6.times.SSC at about 45° C., followed by one or more washes in 0.2×SSC, 0.1% SDS at 65° C., or substantially similar conditions.

“Isolated” (used interchangeably with “substantially pure”), when applied to nucleic acid i.e., polynucleotide sequences that encode VLA antagonists, means an RNA or DNA polynucleotide, portion of genomic polynucleotide, cDNA or synthetic polynucleotide which, by virtue of its origin or manipulation: (i) is not associated with all of a polynucleotide with which it is associated in nature (e.g., is present in a host cell as an expression vector, or a portion thereof); or (ii) is linked to a nucleic acid or other chemical moiety other than that to which it is linked in nature; or (iii) does not occur in nature. By “isolated” it is further meant a polynucleotide sequence that is: (i) amplified in vitro by, for example, polymerase chain reaction (PCR); (ii) synthesized chemically; (iii) produced recombinantly by cloning; or (iv) purified, as by cleavage and gel separation. Thus, “substantially pure nucleic acid” is a nucleic acid which is not immediately contiguous with one or both of the coding sequences with which it is normally contiguous in the naturally occurring genome of the organism from which the nucleic acid is derived. Substantially pure DNA also includes a recombinant DNA which is part of a hybrid gene encoding additional integrin sequences.

“Isolated” (used interchangeably with “substantially pure”), when applied to polypeptides means a polypeptide or a portion thereof which, by virtue of its origin or manipulation: (i) is present in a host cell as the expression product of a portion of an expression vector; or (ii) is linked to a protein or other chemical moiety other than that to which it is linked in nature; or (iii) does not occur in nature, for example, a protein that is chemically manipulated by appending, or adding at least one hydrophobic moiety to the protein so that the protein is in a form not found in nature. By “isolated” it is further meant a protein that is: (i) synthesized chemically; or (ii) expressed in a host cell and purified away from associated and contaminating proteins. The term generally means a polypeptide that has been separated from other proteins and nucleic acids with which it naturally occurs. Preferably, the polypeptide is also separated from substances such as antibodies or gel matrices (polyacrylamide) which are used to purify it.

A “pharmacological agent” is defined as one or more compounds or molecules or other chemical entities administered to a subject (in addition to the VLA-4 antagonists) that affects the action of the antagonist. The term “pharmacological agent” as used herein refers to such an agent(s) that are administered during “combination therapy” where the VLA-4 antagonist is administered either prior to, after, or simultaneously with, administration of one or more pharmacological agents.

“Protein,” as used herein refers to any polymer consisting essentially of any of the 20 amino acids. Although “polypeptide” is often used in reference to relatively large polypeptides, and “peptide” is often used in reference to small polypeptides, usage of these terms in the art overlaps and is varied. The term “protein” as used herein refers to peptides, proteins and polypeptides, unless otherwise noted.

The terms “peptide(s)”, “protein(s)” and “polypeptide(s)” are used interchangeably herein. The terms “polynucleotide sequence” and “nucleotide sequence” are also used interchangeably herein.

“Recombinant,” as used herein, means that a protein is derived from recombinant, mammalian expression systems. Since integrin is not glycosylated nor contains disulfide bonds, it can be expressed in most prokaryotic and eukaryotic expression systems.

“Small molecule” VLA-4 antagonist refers to chemical agents (i.e., organic molecules) capable of disrupting the integrin/integrin ligand interaction by, for instance, blocking VLA-4/VCAM interactions by binding VLA-4 on the surface of cells or binding VCAM-1 on the surface of cells. Such small molecules may also bind respective VLA-4 and VCAM-1 receptors. VLA-4 and VCAM-1 small molecule inhibitors may themselves be peptides, semi-peptidic compounds or non-peptidic compounds, such as small organic molecules that are antagonists of the VCAM-1/VLA-4 interaction.

A VLA-4 antagonist (and a therapeutic composition comprising the same) is said to have “therapeutic efficacy,” and an amount of the agent is said to be “therapeutically effective,” if administration of that amount of the agent is sufficient to cause a clinically significant improvement in neurological recovery in a standard neurological test (see below, Methods of Treatment) when administered to a subject (e.g., an animal model or human patient) after brain damage (e.g., stroke, e.g., ischemic stroke).

The term “treating”, as used herein, refers to administering a therapy in an amount, manner (e.g., schedule of administration), and/or mode (e.g., route of administration), effective to improve a disorder or a symptom thereof, or to prevent or slow the progression of a disorder or a symptom thereof. This can be evidenced by, e.g., an improvement in a parameter associated with a disorder or a symptom thereof, e.g., to a statistically significant degree or to a degree detectable to one skilled in the art. An effective amount, manner, or mode can vary depending on the subject and may be tailored to the subject. By preventing or slowing progression of a disorder or a symptom thereof, a treatment can prevent or slow deterioration resulting from a disorder or a symptom thereof in an affected or diagnosed subject.

The term “biologic” refers to a protein-based therapeutic agent. In a preferred embodiment, the biologic is at least 10, 20, 130, 40, 50 or 100 amino acid residues in length.

A “VLA-4 binding agent” refers to any compound that binds to VLA-4 integrin with a K_{d} of less than 10⁻⁶ M. An example of a VLA-4 binding agent is a VLA-4 binding protein, e.g., a VLA-4 binding antibody such as natalizumab.

A “VLA-4 antagonist” refers to any compound that at least partially inhibits an activity of a VLA-4 integrin, particularly a binding activity of a VLA-4 integrin or a signaling activity, e.g., ability to transduce a VLA-4 mediated signal. For example, a VLA-4 antagonist may inhibit binding of VLA-4 to a cognate ligand of VLA-4, e.g., a cell surface protein such as VCAM-1, or to an extracellular matrix component, such as fibronectin or osteopontin. A typical VLA-4 antagonist can bind to VLA-4 or to a VLA-4 ligand, e.g., VCAM-1 or an extracellular matrix component, such as fibronectin or osteopontin. A VLA-4 antagonist that binds to VLA-4 may bind to either the α4 subunit or the 01 subunit, or to both. A VLA-4 antagonist may also interact with other α4 subunit containing integrins (e.g., α4β7) or with other β1 containing integrins. A VLA-4 antagonist may bind to VLA-4 or to a VLA-4 ligand with a K_{d} of less than 10⁻⁶, 10⁻⁷, 10⁻8, 10⁻⁹, or 10⁻¹⁰ M.

The term “antibody molecule” refers to an antibody or antigen binding fragment thereof. As used herein, the term “antibody” refers to a protein that includes at least one immunoglobulin variable region, e.g., an amino acid sequence that provides an immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab fragments, F(ab′)₂ fragments, Fd fragments, Fv fragments, and dAb fragments) as well as complete antibodies, e.g., intact immunoglobulins of types IgA, IgG, IgE, IgD, IgM (as well as subtypes thereof). The light chains of the immunoglobulin may be of types kappa or lambda. In one embodiment, the antibody is glycosylated. An antibody can be functional for antibody-dependent cytotoxicity and/or complement-mediated cytotoxicity, or may be non-functional for one or both of these activities.

The VH and VL regions can be further subdivided into regions of hypervariability, termed “complementarity determining regions” (“CDR”), interspersed with regions that are more conserved, termed “framework regions” (FR). The extent of the FR's and CDR's has been precisely defined (see, Kabat, E. A., et al. (1991) Sequences of Proteins of Immunological Interest, Fifth Edition, US Department of Health and Human Services, NIH Publication No. 91-3242; and Chothia, C. et al. (1987) J. Mol. Biol. 196:901-917). Kabat definitions are used herein. Each VH and VL is typically composed of three CDR's and four FR's, arranged from amino-terminus to carboxyl-terminus in the following order: FR1, CDR1, FR2, CDR2, FR3, CDR3, FR4.

An “immunoglobulin domain” refers to a domain from the variable or constant domain of immunoglobulin molecules. Immunoglobulin domains typically contain two β-sheets formed of about seven β-strands, and a conserved disulphide bond (see, e.g., A. F. Williams and A. N. Barclay 1988 Ann. Rev Immunol. 6:381-405).

As used herein, an “immunoglobulin variable domain sequence” refers to an amino acid sequence that can form the structure of an immunoglobulin variable domain. For example, the sequence may include all or part of the amino acid sequence of a naturally-occurring variable domain. For example, the sequence may omit one, two or more N- or C-terminal amino acids, internal amino acids, may include one or more insertions or additional terminal amino acids, or may include other alterations. In one embodiment, a polypeptide that includes an immunoglobulin variable domain sequence can associate with another immunoglobulin variable domain sequence to form a target binding structure (or “antigen binding site”), e.g., a structure that interacts with VLA-4.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains. The heavy and light immunoglobulin chains can be connected by disulfide bonds. The heavy chain constant region typically includes three constant domains, CH₁, CH₂ and CH₃. The light chain constant region typically includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (C1q) of the classical complement system.

One or more regions of an antibody can be human, effectively human, or humanized. For example, one or more of the variable regions can be human or effectively human. For example, one or more of the CDRs, e.g., HC CDR1, HC CDR2, HC CDR3, LC CDR1, LC CDR2, and LC CDR3, can be human. Each of the light chain CDRs can be human. HC CDR3 can be human. One or more of the framework regions can be human, e.g., FR1, FR2, FR3, and FR4 of the HC or LC. In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. One or more of the constant regions can be human, effectively human, or humanized. In another embodiment, at least 70, 75, 80, 85, 90, 92, 95, or 98% of the framework regions (e.g., FR1, FR2, and FR3, collectively, or FR1, FR2, FR3, and FR4, collectively) or the entire antibody can be human, effectively human, or humanized. For example, FR1, FR2, and FR3 collectively can be at least 70, 75, 80, 85, 90, 92, 95, 98, or 99% identical to a human sequence encoded by a human germline segment.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

A “humanized” immunoglobulin variable region is an immunoglobulin variable region that is modified such that the modified form elicits less of an immune response in a human than does the non-modified form, e.g., is modified to include a sufficient number of human framework amino acid positions such that the immunoglobulin variable region does not elicit an immunogenic response in a normal human. Descriptions of “humanized” immunoglobulins include, for example, U.S. Pat. No. 6,407,213 and U.S. Pat. No. 5,693,762. In some cases, humanized immunoglobulins can include a non-human amino acid at one or more framework amino acid positions.

All or part of an antibody can be encoded by an immunoglobulin gene or a segment thereof. Exemplary human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 Kd or 214 amino acids) are encoded by a variable region gene at the NH2-terminus (about 110 amino acids) and a kappa or lambda constant region gene at the COOH-terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids).

The term “antigen-binding fragment” of a full length antibody refers to one or more fragments of a full-length antibody that retain the ability to specifically bind to a target of interest, e.g., VLA-4. Examples of binding fragments encompassed within the term “antigen-binding fragment” of a full length antibody include (i) a Fab fragment, a monovalent fragment consisting of the VL, VH, CL and CH1 domains; (ii) a F(ab′).sub.2 fragment, a bivalent fragment including two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:54-546), which consists of a VH domain; and (vi) an isolated complementarity determining region (CDR) that retains functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See, e.g., Bird et al. (1988) Science 242:423-426; and Huston et al. (1988) Proc. Natl. Acad. Sci. USA 85:5879-5883.

Practice of the present invention will employ, unless indicated otherwise, conventional techniques of cell biology, cell culture, molecular biology, microbiology, recombinant DNA, protein chemistry, pharmacology and immunology, which are within the skill of the art. Such techniques are described in the literature. Unless stipulated otherwise, all references cited in the Detailed Description are incorporated herein by reference.

### VLA-4 Antagonists

A VLA-4 antagonist is an antagonist of interactions of α4 integrins with their ligands, such as the VCAM-1/VLA-4 interaction. This is an agent, e.g., a polypeptide or other molecule, which can inhibit or block VCAM-1 and/or VLA-4-mediated binding or which can otherwise modulate VCAM-1 and/or VLA-4 function, e.g., by inhibiting or blocking VLA-4-ligand mediated VLA-4 signal transduction or VCAM-1-ligand mediated VCAM-1 signal transduction and which is effective in the treatment of acute brain injury, preferably in the same manner as anti-VLA-4 binding agents such as anti-VLA-4 antibodies.

A VLA-4 antagonist can have one or more of the following properties: (1) it coats, or binds to, VLA-4 on the surface of a VLA-4 bearing cell (e.g., an endothelial cell) with sufficient specificity to inhibit a VLA-4-ligand/VLA-4 interaction, e.g., the VCAM-1/VLA-4 interaction; (2) it coats, or binds to, VLA-4 on the surface of a VLA-4 bearing cell (i.e., a lymphocyte) with sufficient specificity to modify, and preferably to inhibit, transduction of a VLA-4-mediated signal e.g., VLA-4/VCAM-1-mediated signaling; (3) it coats, or binds to, a VLA-4-ligand, (e.g., VCAM-1) on endothelial cells with sufficient specificity to inhibit the VLA-4/VCAM-1 interaction; (4) it coats, or binds to, a VLA-4-ligand (e.g., VCAM-1) with sufficient specificity to modify, and preferably to inhibit, transduction of VLA-4-ligand mediated VLA-4 signaling, e.g., VCAM-1-mediated VLA-4 signaling. In preferred embodiments the antagonist has one or both of properties 1 and 2. In other preferred embodiments the antagonist has one or both of properties 3 and 4. Moreover, more than one antagonist can be administered to a patient, e.g., an agent which binds to VLA-4 can be combined with an agent which binds to VCAM-1.

For example, antibody molecules as well as soluble forms of the natural binding proteins for VLA-4 and VCAM-1 are useful.

### VLA-4 Antagonist Antibody Molecules

Natalizumab, an α4 integrin binding antibody, inhibits the migration of leukocytes from the blood to the central nervous system. Natalizumab binds to VLA-4 on the surface of activated T-cells and other mononuclear leukocytes. It can disrupt adhesion between the T-cell and endothelial cells, and thus prevent migration of mononuclear leukocytes across the endothelium and into the parenchyma. As a result, the levels of proinflammatory cytokines can also be reduced.

Natalizumab and related VLA-4 binding antibodies are described, e.g., in U.S. Pat. No. 5,840,299. Monoclonal antibodies 21.6 and HP1/2 are exemplary murine monoclonal antibodies that bind VLA-4. Natalizumab is a humanized version of murine monoclonal antibody 21.6 (see, e.g., U.S. Pat. No. 5,840,299). A humanized version of HP1/2 has also been described (see, e.g., U.S. Pat. No. 6,602,503). Several additional VLA-4 binding monoclonal antibodies, such as HP2/1, HP2/4, L25 and P4C2, are described, e.g., in U.S. Pat. No. 6,602,503; Sanchez-Madrid et al., 1986 Eur. J. Immunol., 16:1343-1349; Hemler et al., 1987 J. Biol. Chem. 2:11478-11485; Issekutz and Wykretowicz, 1991, J. Immunol., 147: 109 (TA-2 mab); Pulido et al., 1991 J. Biol. Chem., 266(16):10241-10245; and U.S. Pat. No. 5,888,507.

Some VLA-4 binding antibody molecules recognize epitopes of the α4 subunit that are involved in binding to a cognate ligand, e.g., VCAM-1 or fibronectin. Many such antibody molecules inhibit binding of VLA-4 to cognate ligands (e.g., VCAM-1 and fibronectin).

Some useful VLA-4 binding antibodies can interact with VLA-4 on cells, e.g., lymphocytes, but do not cause cell aggregation. However, other VLA-4 binding antibodies have been observed to cause such aggregation. HP1/2 does not cause cell aggregation. The HP1/2 monoclonal antibody (Sanchez-Madrid et al., 1986) has an extremely high potency, blocks VLA-4 interaction with both VCAM1 and fibronectin, and has the specificity for epitope B on VLA-4. This antibody and other B epitope-specific antibodies (such as B1 or B2 epitope binding antibodies; Pulido et al., 1991, supra) represent one class of VLA-4 binding antibodies that can be used in the methods described herein. Antibodies that compete for binding with a VLA-4 binding antibody, e.g., natalizumab, can also be used in the methods described herein.

An exemplary VLA-4 binding antibody molecule has one or more CDRs, e.g., all three HC CDRs and/or all three LC CDRs of a particular antibody disclosed herein, or CDRs that are, in sum, at least 80, 85, 90, 92, 94, 95, 96, 97, 98, 99% identical to such an antibody, e.g., natalizumab. In one embodiment, the H1 and H2 hypervariable loops have the same canonical structure as those of an antibody described herein. In one embodiment, the L1 and L2 hypervariable loops have the same canonical structure as those of an antibody molecule described herein.

In one embodiment, the amino acid sequence of the HC and/or LC variable domain sequence is at least 70, 80, 85, 90, 92, 95, 97, 98, 99, or 100% identical to the amino acid sequence of the HC and/or LC variable domain of an antibody described herein, e.g., natalizumab. The amino acid sequence of the HC and/or LC variable domain sequence can differ by at least one amino acid, but no more than ten, eight, six, five, four, three, or two amino acids from the corresponding sequence of an antibody described herein, e.g., natalizumab. For example, the differences may be primarily or entirely in the framework regions.

The amino acid sequences of the HC and LC variable domain sequences can be encoded by a nucleic acid sequence that hybridizes under high stringency conditions to a nucleic acid sequence described herein or one that encodes a variable domain or an amino acid sequence described herein. In one embodiment, the amino acid sequences of one or more framework regions (e.g., FR1, FR2, FR3, and/or FR4) of the HC and/or LC variable domain are at least 70, 80, 85, 90, 92, 95, 97, 98, 99, or 100% identical to corresponding framework regions of the HC and LC variable domains of an antibody described herein. In one embodiment, one or more heavy or light chain framework regions (e.g., HC FR1, FR2, and FR3) are at least 70, 80, 85, 90, 95, 96, 97, 98, or 100% identical to the sequence of corresponding framework regions from a human germline antibody.

### Other VLA-4 Antagonist Polypeptides

In some embodiments, the VLA-4 antagonist can be a soluble form of a ligand. Soluble forms of the ligand proteins include soluble VCAM-I or fibronectin peptides, VCAM-I fusion proteins, or bifunctional VCAM-I/Ig fusion proteins. For example, a soluble form of a VLA-4 ligand or a fragment thereof may be administered to bind to VLA-4, and in some instances, compete for a VLA-4 binding site on cells, thereby leading to effects similar to the administration of antagonists such as anti-VLA-4 antibodies. For example, soluble VLA-4 integrin mutants that bind VLA-4 ligand but do not elicit integrin-dependent signaling are suitable for use in the described methods. Such mutants can act as competitive inhibitors of wild type integrin protein and are considered “antagonists.” Soluble forms of the natural binding proteins for VLA-4 include soluble VCAM-1 peptides, VCAM-1 fusion proteins, bifunctional VCAM-1/lg fusion proteins (e.g. “chimeric” molecules, discussed above), fibronectin, fibronectin having an alternatively spliced non-type III connecting segment, and fibronectin peptides containing the amino acid sequence EILDV or a similar conservatively substituted amino acid sequence. As used herein, a “soluble VLA-4 peptide” or a “soluble VCAM-1 peptide” is an VLA4 or VCAM-1 polypeptide incapable of anchoring itself in a membrane. Such soluble polypeptides include, for example, VLA-4 and VCAM polypeptides that lack a sufficient portion of their membrane spanning domain to anchor the polypeptide or are modified such that the membrane spanning domain is non-functional. These binding agents can act by competing with the cell-surface binding protein for VLA-4 or by otherwise altering VLA-4 function. For example, a soluble form of VCAM-1 (see, e.g., Osborn et al. 1989, Cell, 59: 1203-1211) or a fragment thereof may be administered to bind to VLA-4, and preferably compete for a VLA-4 binding site on VCAM-1-bearing cells, thereby leading to effects similar to the administration of antagonists such as small molecules or anti-VLA-4 antibodies.

Small Molecule VLA-4 Antagonists

“Small molecules” are agents that mimic the action of peptides to disrupt VLA-4/ligand interactions by, for instance, binding VLA-4 and blocking interaction with a VLA-4 ligand (e.g., VCAM-I or fibronectin), or by binding a VLA-4 ligand and preventing the ligand from interacting with VLA-4. One exemplary small molecule is an oligosaccharide that mimics the binding domain of a VLA-4 ligand (e.g., fibronectin or VCAM-I) and binds the ligand-binding domain of VLA-4. (See, Devlin et al., Science 249: 400-406 (1990); Scott and Smith, Science 249:386-390 (1990); and U.S. Pat. No. 4,833,092 (Geysen), all incorporated herein by reference.)

A “small molecule” may be chemical compound, e.g., an organic compound, or a small peptide, or a larger peptide-containing organic compound or non-peptidic organic compound. A “small molecule” is not intended to encompass an antibody or antibody fragment. Although the molecular weight of small molecules is generally less than 2000 Daltons, this figure is not intended as an absolute upper limit on molecular weight.

Examples of other small molecules useful in the invention can be found in Komoriya et al. (“The Minimal Essential Sequence for a Major Cell Type-Specific Adhesion Site (CS1) Within the Alternatively Spliced Type III Connecting Segment Domain of Fibronectin Is Leucine-Aspartic Acid-Valine”, J. Biol. Chem., 266 (23), pp. 15075-79 (1991)). They identified the minimum active amino acid sequence necessary to bind VLA-4 and synthesized a variety of overlapping peptides based on the amino acid sequence of the CS-1 region (the VLA-4 binding domain) of a particular species of fibronectin. They identified an 8-amino acid peptide, Glu-Ile-Leu-Asp-Val-Pro-Ser-Thr, as well as two smaller overlapping pentapeptides, Glu-Ile-Leu-Asp-Val and Leu-Asp-Val-Pro-Ser, that possessed inhibitory activity against fibronectin-dependent cell adhesion. Certain larger peptides containing the LDV sequence were subsequently shown to be active in vivo (T. A. Ferguson et al., “Two integrin Binding Peptides Abrogate T-cell-Mediated Immune Responses In Vivo”, Proc. Natl. Acad. Sci. USA, 88, pp. 8072-76 (1991); and S. M. Wahl et al., “Synthetic Fibronectin Peptides Suppress Arthritis in Rats by Interrupting Leukocyte Adhesion and Recruitment”, J. Clin. Invest., 94, pp. 655-62 (1994)). A cyclic pentapeptide, Arg-Cys-Asp-TPro-Cys (wherein TPro denotes 4-thioproline), which can inhibit both VLA-4 and VLA-5 adhesion to fibronectin has also been described. (See, e.g., D. M. Nowlin et al. “A Novel Cyclic Pentapeptide Inhibits Alpha4Beta1 Integrin-mediated Cell Adhesion”, J. Biol. Chem., 268(27), pp. 20352-59 (1993); and PCT publication PCT/US91/04862). This pentapeptide was based on the tripeptide sequence Arg-Gly-Asp from fibronectin which had been known as a common motif in the recognition site for several extracellular-matrix proteins. Examples of other VLA-4 inhibitors have been reported, for example, in Adams et al. “Cell Adhesion Inhibitors”, PCT US97/13013, describing linear peptidyl compounds containing beta-amino acids which have cell adhesion inhibitory activity. International patent applications WO 94/15958 and WO 92/00995 describe cyclic peptide and peptidomimetic compounds with cell adhesion inhibitory activity. International patent applications WO 93/08823 and WO 92/08464 describe guanidinyl-, urea- and thiourea-containing cell adhesion inhibitory compounds. U.S. Pat. No. 5,260,277 describes guanidinyl cell adhesion modulation compounds. Other peptidyl antagonists of VLA-4 have been described in D. Y. Jackson et al., “Potent α4β1 peptide antagonists as potential anti-inflammatory agents”, J. Med. Chem., 40,3359 (1997); H. Shroff et al., “Small peptide inhibitors of α4β7 mediated MadCAM-1 adhesion to lymphocytes”, Bio. Med, Chem. Lett., 1 2495 (1996); U.S. Pat. No. 5,510,332, PCT Publications WO 98/53814, WO97/03094, WO97/02289, WO96/40781, WO96/22966, WO96/20216, WO96/01644, WO96106108, and WO95/15973, and others.

Such small molecule agents may be produced by synthesizing a plurality of peptides (e.g., 5 to 20 amino acids in length), semi-peptidic compounds or non-peptidic, organic compounds, and then screening those compounds for their ability to inhibit the VLA-4/VCAM interaction. See generally U.S. Pat. No. 4,833,092, Scott and Smith, “Searching for Peptide Ligands with an Epitope Library”, Science, 249, pp. 386-90 (1990), and Devlin et al., “Random Peptide Libraries: A Source of Specific Protein Binding Molecules”, Science, 249, pp. 40407 (1990).

### Antibody Generation

Antibodies that bind to VLA-4 can be generated by immunization, e.g., using an animal, or by in vitro methods such as phage display. All or part of VLA-4 can be used as an immunogen. For example, the extracellular region of the α4 subunit can be used as an immunogen. In one embodiment, the immunized animal contains immunoglobulin producing cells with natural, human, or partially human immunoglobulin loci. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XenoMouse™, Green et al., Nature Genetics 7:13-21 (1994), US 2003-0070185, U.S. Pat. No. 5,789,650, and WO 96/34096.

Non-human antibodies to VLA-4 can also be produced, e.g., in a rodent. The non-human antibody can be humanized, e.g., as described in U.S. Pat. No. 6,602,503, EP 239 400, U.S. Pat. No. 5,693,761, and U.S. Pat. No. 6,407,213.

EP 239 400 (Winter et al.) describes altering antibodies by substitution (within a given variable region) of their complementarity determining regions (CDRs) for one species with those from another. CDR-substituted antibodies can be less likely to elicit an immune response in humans compared to true chimeric antibodies because the CDR-substituted antibodies contain considerably less non-human components (Riechmann et al., 1988, Nature 332, 323-327; Verhoeyen et al., 1988, Science 239, 1534-1536). Typically, CDRs of a murine antibody substituted into the corresponding regions in a human antibody by using recombinant nucleic acid technology to produce sequences encoding the desired substituted antibody. Human constant region gene segments of the desired isotype (usually gamma I for CH and kappa for CL) can be added and the humanized heavy and light chain genes can be co-expressed in mammalian cells to produce soluble humanized antibody.

Queen et al. (Proc. Natl. Acad. Sci. U.S.A. 86:10029-33, 1989) and WO 90/07861 have described a process that includes choosing human V framework regions by computer analysis for optimal protein sequence homology to the V region framework of the original murine antibody, and modeling the tertiary structure of the murine V region to visualize framework amino acid residues that are likely to interact with the murine CDRs. These murine amino acid residues are then superimposed on the homologous human framework. See also U.S. Pat. Nos. 5,693,762; 5,693,761; 5,585,089; and 5,530,101. Tempest et al., 1991, Biotechnology 9:266-271, utilize, as standard, the V region frameworks derived from NEWM and REI heavy and light chains, respectively, for CDR-grafting without radical introduction of mouse residues. An advantage of using the Tempest et al. approach to construct NEWM and REI based humanized antibodies is that the three dimensional structures of NEWM and REI variable regions are known from x-ray crystallography and thus specific interactions between CDRs and V region framework residues can be modeled.

Non-human antibodies can be modified to include substitutions that insert human immunoglobulin sequences, e.g., consensus human amino acid residues at particular positions, e.g., at one or more (preferably at least five, ten, twelve, or all) of the following positions: (in the

FR of the variable domain of the light chain) 4L, 35L, 36L, 38L, 43L, 44L, 58L, 46L, 62L, 63L, 64L, 65L, 66L, 67L, 68L, 69L, 70L, 71L, 73L, 85L, 87L, 98L, and/or (in the FR of the variable domain of the heavy chain) 2H, 4H, 24H, 36H, 37H, 39H, 43H, 45H, 49H, 58H, 60H, 67H, 68H, 69H, 70H, 73H, 74H, 75H, 78H, 91H, 92H, 93H, and/or 103H (according to the Kabat numbering). See, e.g., U.S. Pat. No. 6,407,213.

Fully human monoclonal antibodies that bind to VLA-4 can be produced, e.g., using in vitro-primed human splenocytes, as described by Boerner et al., 1991, J. Immunol., 147, 86-95. They may be prepared by repertoire cloning as described by Persson et al., 1991, Proc. Nat. Acad. Sci. USA, 88: 2432-2436 or by Huang and Stollar, 1991, J. Immunol. Methods 141, 227-236; also U.S. Pat. No. 5,798,230. Large nonimmunized human phage display libraries may also be used to isolate high affinity antibodies that can be developed as human therapeutics using standard phage technology (see, e.g., Vaughan et al, 1996; Hoogenboom et al. (1998) Immunotechnology 4:1-20; and Hoogenboom et al. (2000) Immunol Today 2:371-8; US 2003-0232333). Transgenic animals, e.g., transgenic mice, expressing human antibody gene sequences may be used to produce human monoclonal antibodies using technology as described in, e.g., Lonberg N. (2005) Nat. Biotechnol. 23(9):1117-25.

### Antibody Production

Antibodies can be produced in prokaryotic and eukaryotic cells. In one embodiment, the antibodies (e.g., scFv's) are expressed in a yeast cell such as Pichia (see, e.g., Powers et al. (2001) J Immunol Methods. 251:123-35), Hanseula, or Saccharomyces.

In one embodiment, antibodies, particularly full length antibodies, e.g., IgG's, are produced in mammalian cells. Exemplary mammalian host cells for recombinant expression include Chinese Hamster Ovary (CHO cells) (including dhfr− CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NS0 myeloma cells and SP2 cells, COS cells, K562, and a cell from a transgenic animal, e.g., a transgenic mammal. For example, the cell is a mammary epithelial cell.

In addition to the nucleic acid sequence encoding the immunoglobulin domain, the recombinant expression vectors may carry additional nucleic acid sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017). Exemplary selectable marker genes include the dihydrofolate reductase (DHFR) gene (for use in dhfr⁻ host cells with methotrexate selection/amplification) and the neo gene (for G418 selection).

In an exemplary system for recombinant expression of an antibody (e.g., a fall length antibody or an antigen-binding portion thereof), a recombinant expression vector encoding both the antibody heavy chain and the antibody light chain is introduced into dhfr⁻ CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, to transfect the host cells, to select for transformants, to culture the host cells, and to recover the antibody from the culture medium. For example, some antibodies can be isolated by affinity chromatography with a Protein A or Protein G.

Antibodies may also include modifications, e.g., modifications that alter Fc function, e.g., to decrease or remove interaction with an Fc receptor or with C1q, or both. For example, the human IgG1 constant region can be mutated at one or more residues, e.g., one or more of residues 234 and 237, e.g., according to the numbering in U.S. Pat. No. 5,648,260. Other exemplary modifications include those described in U.S. Pat. No. 5,648,260.

For some antibodies that include an Fc domain, the antibody production system may be designed to synthesize antibodies in which the Fc region is glycosylated. For example, the Fc domain of IgG molecules is glycosylated at asparagine 297 in the CH2 domain. This asparagine is the site for modification with biantennary-type oligosaccharides. This glycosylation participates in effector functions mediated by Fcγ receptors and complement C1q (Burton and Woof (1992) Adv. Immunol. 51:1-84; Jefferis et al. (1998) Immunol. Rev. 163:59-76). The Fc domain can be produced in a mammalian expression system that appropriately glycosylates the residue corresponding to asparagine 297. The Fc domain can also include other eukaryotic post-translational modifications.

Antibodies can also be produced by a transgenic animal. For example, U.S. Pat. No. 5,849,992 describes a method for expressing an antibody in the mammary gland of a transgenic mammal. A transgene is constructed that includes a milk-specific promoter and nucleic acid sequences encoding the antibody of interest, e.g., an antibody described herein, and a signal sequence for secretion. The milk produced by females of such transgenic mammals includes, secreted-therein, the antibody of interest, e.g., an antibody described herein. The antibody can be purified from the milk, or for some applications, used directly.

Antibodies can be modified, e.g., with a moiety that improves its stabilization and/or retention in circulation, e.g., in blood, serum, lymph, bronchoalveolar lavage, or other tissues, e.g., by at least 1.5, 2, 5, 10, or 50 fold.

For example, a VLA-4 binding antibody can be associated with a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 daltons (or about 1,000 to about 15,000, and 2,000 to about 12,500) can be used.

For example, a VLA-4 binding antibody can be conjugated to a water soluble polymer, e.g., a hydrophilic polyvinyl polymer, e.g. polyvinylalcohol or polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; branched or unbranched polysaccharides that comprise the saccharide monomers D-mannose, D- and L-galactose, fucose, fructose, D-xylose, L-arabinose, D-glucuronic acid, sialic acid, D-galacturonic acid, D-mannuronic acid (e.g. polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextrane sulfate, dextran, dextrins, glycogen, or the polysaccharide subunit of acid mucopolysaccharides, e.g., hyaluronic acid; polymers of sugar alcohols such as polysorbitol and polymannitol; heparin or heparon.

### Pharmaceutical Compositions

A VLA-4 antagonist, e.g., a VLA-4 binding agent, such as a VLA-4 binding antibody, (e.g., natalizumab) can be formulated as a pharmaceutical composition. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible.

A “pharmaceutically acceptable salt” refers to a salt that retains the desired biological activity of the parent compound and does not impart any undesired toxicological effects (see, e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

VLA-4 antagonists, e.g., a VLA-4 binding antibody, e.g., natalizumab, and other agents described herein can be formulated according to standard methods. Exemplary pharmaceutical formulation is described in Gennaro (ed.), Remington: The Science and Practice of Pharmacy, 20.sup.th ed., Lippincott, Williams & Wilkins (2000) (ISBN: 0683306472); Ansel et al., Pharmaceutical Dosage Forms and Drug Delivery Systems, 7.sup.th Ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X).

In one embodiment, a VLA-4 antagonist, e.g., a VLA-4 binding antibody, e.g., natalizumab or another agent (e.g., another antibody) can be formulated with excipient materials, such as sodium chloride, sodium dibasic phosphate heptahydrate, sodium monobasic phosphate, and polysorbate 80. It can be provided, for example, in a buffered solution at a concentration of about 20 mg/ml and can be stored at 2-8° C. Natalizumab can be formulated as described on the manufacturer's label.

Pharmaceutical compositions may also be in a variety of other forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically compositions for the agents described herein are in the form of injectable or infusible solutions.

Such compositions can be administered by a parenteral mode (e.g., intravenous, subcutaneous, intraperitoneal, or intramuscular injection). The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Pharmaceutical compositions typically must be sterile and stable under the conditions of manufacture and storage. A pharmaceutical composition can also be tested to insure it meets regulatory and industry standards for administration.

The composition can be formulated as a solution, microemulsion, dispersion, liposome, or other ordered structure suitable to high drug concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

### Administration

A VLA-4 antagonist, e.g., a VLA-4 binding antibody can be administered to a subject, e.g., a human subject, by a variety of methods. For many applications, the route of administration is one of: intravenous injection or infusion, subcutaneous injection, or intramuscular injection. A VLA-4 binding antibody, such as natalizumab, can be administered as a fixed dose, or in a mg/kg dose, but preferably as a fixed dose. The antibody can be administered intravenously (IV) or subcutaneously (SC).

The antibody, e.g., natalizumab, is typically administered at a fixed unit dose of between 50-1000 mg IV, e.g., between 100-600 mg IV, e.g., between 200 and 400 mg IV, e.g., about 300 mg IV. When administered subcutaneously, the antibody is typically administered at a dose between 50-100 mg SC (e.g., 75 mg). It can also be administered in a bolus at a dose of between 1 and 10 mg/kg, e.g., about 6.0, 4.0, 3.0, 2.0, 1.0 mg/kg. In some cases, continuous administration may be indicated, e.g., via a subcutaneous pump.

The VLA-4 antagonist, e.g., the VLA-4 binding antibody molecule, is administered to a subject within 12 hours or less, e.g., 11, 10, 9, 8, 7, 6, 5, 4, 3, 2 hours or less after the onset of a stroke. Preferably, the VLA-4 antagonist, e.g., the VLA-4 binding antibody molecule, e.g., natalizumab, is administered within 9 hours or less, e.g., approximately 1 to 9 hours, e.g., 9 to 2 hours, e.g., 9 to 3 hours, e.g., 9 to 4 hours, e.g., 9 to 5 hours, after the onset of a stroke. In preferred embodiments, the VLA-4 antagonist, e.g., the VLA-4 binding antibody molecule, e.g., natalizumab, is administered within 6 hours or less, e.g., 6, 5, 4, 3, 2 or 1 hour after the onset of a stroke.

In some embodiments, the VLA-4 antagonist, e.g., the VLA-4 binding antibody molecule, e.g., natalizumab, is administered at a dose of between 200 and 400 mg within 12 hours or less, e.g., 11, 10, 9, 8, 7, 6, 5, 4, 3, 2 hours or less after the onset of a stroke. In some embodiments, the VLA-4 antagonist, e.g., the VLA-4 binding antibody molecule, e.g., natalizumab, is administered at a dose of between 200 and 400 mg within 9 hours or less, e.g., approximately 1 to 9 hours, e.g., 9 to 2 hours, e.g., 9 to 3 hours, e.g., 9 to 4 hours, e.g., 9 to 5 hours, after the onset of a stroke. In some embodiments, the VLA-4 antagonist, e.g., the VLA-4 binding antibody molecule, e.g., natalizumab, is administered at a dose of between 200 and 400 mg within 6 hours or less, e.g., 6, 5, 4, 3, 2 or 1 hour after the onset of a stroke.

In some embodiments, the VLA-4 antagonist, e.g., the VLA-4 binding antibody molecule, e.g., natalizumab, is administered at a dose of about 300 mg within 12 hours or less, e.g., 11, 10, 9, 8, 7, 6, 5, 4, 3, 2 hours or less after the onset of a stroke. In some embodiments, the VLA-4 antagonist, e.g., the VLA-4 binding antibody molecule, e.g., natalizumab, is administered at a dose of about 300 mg within 9 hours or less, e.g., approximately 1 to 9 hours, e.g., 9 to 2 hours, e.g., 9 to 3 hours, e.g., 9 to 4 hours, e.g., 9 to 5 hours, after the onset of a stroke. In some embodiments, the VLA-4 antagonist, e.g., the VLA-4 binding antibody molecule, e.g., natalizumab, is administered at a dose of about 300 mg within 6 hours or less, e.g., 6, 5, 4, 3, 2 or 1 hour after the onset of a stroke. In some embodiments, the VLA-4 antagonist, e.g., the VLA-4 binding antibody molecule, e.g., natalizumab, is administered at a dose of 300 mg within 6 hours after the onset of a stroke.

In some embodiments, the VLA-4 antagonist, e.g., the VLA-4 binding antibody molecule, e.g., natalizumab, is administered at a dose of between 150 and 450 mg within 12 hours or less, e.g., 11, 10, 9, 8, 7, 6, 5, 4, 3, 2 hours or less after the onset of a stroke. In some embodiments, the VLA-4 antagonist, e.g., the VLA-4 binding antibody molecule, e.g., natalizumab, is administered at a dose of between 150 and 450 mg within 9 hours or less, e.g., approximately 1 to 9 hours, e.g., 9 to 2 hours, e.g., 9 to 3 hours, e.g., 9 to 4 hours, e.g., 9 to 5 hours, after the onset of a stroke. In some embodiments, the VLA-4 antagonist, e.g., the VLA-4 binding antibody molecule, e.g., natalizumab, is administered at a dose of between 150 and 450 mg within 6 hours or less, e.g., 6, 5, 4, 3, 2 or 1 hour after the onset of a stroke.

In some embodiments, the VLA-4 antagonist, e.g., the VLA-4 binding antibody molecule, e.g., natalizumab, is administered at a dose of about 150 mg within 12 hours or less, e.g., 11, 10, 9, 8, 7, 6, 5, 4, 3, 2 hours or less after the onset of a stroke. In some embodiments, the VLA-4 antagonist, e.g., the VLA-4 binding antibody molecule, e.g., natalizumab, is administered at a dose of about 150 mg within 9 hours or less, e.g., approximately 1 to 9 hours, e.g., 9 to 2 hours, e.g., 9 to 3 hours, e.g., 9 to 4 hours, e.g., 9 to 5 hours, after the onset of a stroke. In some embodiments, the VLA-4 antagonist, e.g., the VLA-4 binding antibody molecule, e.g., natalizumab, is administered at a dose of about 150 mg within 6 hours or less, e.g., 6, 5, 4, 3, 2 or 1 hour after the onset of a stroke. In some embodiments, the VLA-4 antagonist, e.g., the VLA-4 binding antibody molecule, e.g., natalizumab, is administered at a dose of 150 mg within 6 hours after the onset of a stroke.

In some embodiments, the VLA-4 antagonist, e.g., the VLA-4 binding antibody molecule, e.g., natalizumab, is administered at a dose of about 450 mg within 12 hours or less, e.g., 11, 10, 9, 8, 7, 6, 5, 4, 3, 2 hours or less after the onset of a stroke. In some embodiments, the VLA-4 antagonist, e.g., the VLA-4 binding antibody molecule, e.g., natalizumab, is administered at a dose of about 450 mg within 9 hours or less, e.g., approximately 1 to 9 hours, e.g., 9 to 2 hours, e.g., 9 to 3 hours, e.g., 9 to 4 hours, e.g., 9 to 5 hours, after the onset of a stroke. In some embodiments, the VLA-4 antagonist, e.g., the VLA-4 binding antibody molecule, e.g., natalizumab, is administered at a dose of about 450 mg within 6 hours or less, e.g., 6, 5, 4, 3, 2 or 1 hour after the onset of a stroke. In some embodiments, the VLA-4 antagonist, e.g., the VLA-4 binding antibody molecule, e.g., natalizumab, is administered at a dose of 450 mg within 6 hours after the onset of a stroke.

The dose can also be chosen to reduce or avoid production of antibodies against the

VLA-4 binding antibody, to achieve greater than 40, 50, 70, 75, or 80% saturation of the α4 subunit, to achieve to less than 80, 70, 60, 50, or 40% saturation of the α4 subunit, or to prevent an increase the level of circulating white blood cells.

In certain embodiments, the active agent may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978.

Pharmaceutical compositions can be administered with medical devices. For example, pharmaceutical compositions can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Of course, many other such implants, delivery systems, and modules are also known.

Dosage unit form or “fixed dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent.

A pharmaceutical composition may include a “therapeutically effective amount” of an agent described herein. A therapeutically effective amount of an agent may also vary according to factors such as the disease state, age, sex, and weight of the individual, and the ability of the compound to elicit a desired response in the individual, e.g., amelioration of at least one disorder parameter, e.g., a parameter of a stroke scale, or amelioration of at least one symptom of the disorder, e.g., stroke. A therapeutically effective amount is also one in which any toxic or a detrimental effect of the composition is outweighed by the therapeutically beneficial effects.

Methods described herein can also include administering a VLA-4 antagonist in combination with another therapeutic modality, e.g., an additional agent (e.g., a pharmacological agent) or a procedure. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The VLA-4 antagonist and the at least one additional therapeutic agent can be administered simultaneously, in the same or in separate compositions, or sequentially. For sequential administration, the antagonist can be administered first, and the additional agent can be administered second, or the order of administration can be reversed.

The additional agent is preferably an agent with some degree of therapeutic efficacy in treating acute brain injury. Such agents may include, but are not limited to, thrombolytic agents such as plasminogen, tissue plasminogen activator (t-PA) or urokinase, agents that target excitotoxic mechanisms such as Selfotel™ or Aptiganel™, agents that target nitric oxide associated neuronal damage such as Lubeluzole™, agents that target ischemia associated neuronal cellular membrane damage such as Tirilizad™, agents that target anti-inflammatory mechanisms such as Enlimomab™. The agent may be combined with the VLA-4 antagonists either prior to, during, or after administration of the antagonists.

### Methods of Treatment

The methods of treatment described herein include administering to a subject suffering from an injury to the central nervous system (e.g., a stroke) an effective amount of a VLA-4 antagonist. The methods include, e.g., administering a VLA-4 antagonist in a specified period from the onset of the injury. For example, the methods include administering the VLA-4 antagonist within 9 hours or less, e.g., 8, 7, 6, 5 hours or less after the onset of the injury. The methods also include administering the VLA-4 antagonist within 4 to 9 hours, e.g., 5 to 8 hours, e.g., 6 to 7 hours, after the onset of the injury. Treating includes administering an amount effective to alleviate, relieve, alter, remedy, ameliorate, improve or affect the disorder, the symptoms of the disorder or the predisposition toward the disorder. The treatment may also delay onset, e.g., prevent onset, or prevent deterioration of a disease or condition.

Preferred methods include treating a subject suffering from an acute middle cerebral artery (MCA) ischemic event or stroke, e.g., ischemic stroke. Ischemic stroke is the rapidly developing loss of brain function(s) due to disturbance in the blood supply to the brain due to ischemia (lack of glucose and oxygen supply) caused by thrombosis (e.g., venous thrombosis), embolism, or systemic hypoperfusion. As a result, the affected area of the brain is unable to function, leading to inability to move one or more limbs on one side of the body, inability to understand or formulate speech, or inability to see one side of the visual field. A stroke is a medical emergency and can cause permanent neurological damage, complications, and/or death.

Symptoms of acute middle cerebral artery (MCA) ischemic event or ischemic stroke include, e.g., hemiplegia, decreased sensation and muscle weakness of the face, numbness, reduction in sensory or vibratory sensation, altered smell, taste, hearing or vision (total or partial), drooping of eyelid (ptosis) and weakness of ocular muscles, decreased reflexes, balance problems and nystagmus, altered breathing and heart rate, weakness in sternocleidomastoid muscle with inability to turn head to one side, weakness in tongue (inability to protrude and/or move from side to side), aphasia, apraxia, visual field defect, memory deficits, hemineglect, disorganized thinking, confusion, hypersexual gestures, anosognosia, trouble walking, altered movement coordination, and vertigo and/or disequilibrium.

Ischemic event or stroke, e.g., ischemic stroke, onset time may be determined by any available method. For example, a subject may questioned, e.g., by a physician, regarding various symptoms of stroke, e.g., as described herein, to identify the approximate time of stroke onset. In some cases, stroke onset time is difficult to pinpoint, such as when a subject awakens with stroke, or if the start of symptoms are otherwise undetectable. In such cases, stroke onset may be determined by identifying the time the subject was last known to be well, e.g., last known normal (LKN). In some cases, MRI of the brain can be used to determine onset time and/or stroke duration in a subject (see, e.g., Petkova et al.; Radiology (2010) MR imaging helps predict time from symptom onset in patients with acute stroke: implications for patients with unknown onset time 257(3):782-92).

Therapies used to treat stroke can also include, e.g., thrombolysis (e.g., tissue plasminogen activator (tPA)), thrombectomy, angioplasty and stenting, therapeutic hypothermia, and medications (e.g., aspirin, clopidogrel and dipyridamole).

The disclosure provides methods of treating (e.g., stabilizing, reducing, or eliminating one or more symptoms or stabilizing the subject's score on a stroke scale) stroke, e.g., acute ischemic stroke, by administering a VLA-4 antagonist to a subject having or suspected of having a stroke). The disclosure also provides methods of preventing stroke or a symptom thereof by administering a VLA-4 antagonist to a subject at risk of developing a stroke (e.g., a subject that has experienced systemic hypoperfusion).

Standard tests for neurological recovery (e.g., National Institute of Health Stroke Scale (NIHSS), Barthel Index, modified Rankin Scale (mRS), Glasgow Outcome Scale, Montreal Cognative Assessment (MoCA), Stroke Impact Scale (SIS-16)) can be employed by skilled artisans to determine efficacy. The NIHSS classifies the severity of a stroke based on a subject's ability to answer questions and perform activities relating to level of consciousness, language, visual-field loss, extraocular movement, motor strength, ataxia, dysarthria, sensory loss and extinction and inattention. There are 15 items and ratings for each item are scored with 3 to 5 grades with 0 as normal and a maximum severity score of 42 for all items. A NIHSS of 1-4 is indicative of a minor stroke; a score of 5-15 is indicative of a moderate stroke, a score of 16-20 is indicative of a moderate to severe stroke; and a score of 21-42 is indicative of a severe stroke.

Additionally provided are methods of treating stroke by administering a VLA-4 antagonist in combination with a second therapy, e.g., thrombolysis (e.g., tissue plasminogen activator (tPA)), thrombectomy, angioplasty and stenting, therapeutic hypothermia, and/or a medication (e.g., aspirin, clopidogrel and dipyridamole). In preferred embodiments, the second therapy is, e.g., a thrombolytic agent, a neuroprotective agent, an anti-inflammatory agent, a steroid, a cytokine or a growth factor. The thrombolytic agent used can be tissue plasminogen activator or urokinase. The neuroprotective agent used can be an agonist to a receptor selected from the group consisting of: N-Methyl-D aspartate receptor (NMDA), a-amino-3-hydroxy-5-methyl-4-isoxazoleproprionic acid receptor (AMPA), glycine receptor, calcium channel receptor, bradykinin B2 receptor and sodium channel receptor, or from the group consisting of: the bradykinin B1 receptor, α-amino butyric acid (GABA) receptor, and Adenosine A1 receptor. Anti-inflammatory agents for use can be interleukin-1 and tumor necrosis factor family members.

### Kits

A VLA-4 antagonist described herein may be provided in a kit. The kit includes a VLA-4 antagonist described herein and, optionally, a container, a pharmaceutically acceptable carrier and/or informational material. The informational material can be descriptive, instructional, marketing or other material that relates to the methods described herein and/or the use of the α4 antagonist for the methods described herein.

The informational material of the kits is not limited in its form. In one embodiment, the informational material can include information about production of the VLA-4 antagonist, physical properties of the α4 antagonist, concentration, date of expiration, batch or production site information, and so forth. In one embodiment, the informational material relates to methods for administering the VLA-4 antagonist, e.g., by a route of administration described herein and/or at a dose and/or dosing schedule described herein.

In one embodiment, the informational material can include instructions to administer a

VLA-4 antagonist described herein in a suitable manner to perform the methods described herein, e.g., in a suitable dose, dosage form, or mode of administration (e.g., a dose, dosage form, or mode of administration described herein). In another embodiment, the informational material can include instructions to administer a VLA-4 antagonist to a suitable subject, e.g., a human, e.g., a human having a stroke, e.g., within 9 hours or less, e.g., 8, 7, 6, 5 or less hours after the onset of a stroke.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about an α4 antagonist described herein and/or its use in the methods described herein. The informational material can also be provided in any combination of formats.

In addition to an α4 antagonist, the composition of the kit can include other ingredients, such as a surfactant, a lyoprotectant or stabilizer, an antioxidant, an antibacterial agent, a bulking agent, a chelating agent, an inert gas, a tonicity agent and/or a viscosity agent, a solvent or buffer, a stabilizer, a preservative, a pharmaceutically acceptable carrier and/or a second agent for treating a condition or disorder described herein. Alternatively, the other ingredients can be included in the kit, but in different compositions or containers than an α4 antagonist described herein.

In some embodiments, a component of the kit is stored in a sealed vial, e.g., with a rubber or silicone closure (e.g., a polybutadiene or polyisoprene closure). In some embodiments, a component of the kit is stored under inert conditions (e.g., under Nitrogen or another inert gas such as Argon). In some embodiments, a component of the kit is stored under anhydrous conditions (e.g., with a desiccant). In some embodiments, a component of the kit is stored in a light blocking container such as an amber vial.

A VLA-4 antagonist described herein can be provided in any form, e.g., liquid, frozen, dried or lyophilized form. It is preferred that a composition including the VLA-4 antagonist described herein be substantially pure and/or sterile. When a VLA-4 antagonist described herein such as natalizumab is provided in a liquid solution, the liquid solution preferably is an aqueous solution, with a sterile aqueous solution being preferred. In one embodiment, the VLA-4 antagonist is supplied with a diluents or instructions for dilution. The diluent can include for example, a salt or saline solution, e.g., a sodium chloride solution having a pH between 6 and 9, lactated Ringer's injection solution, D5W, or PLASMA-LYTE A Injection pH 7.4® (Baxter, Deerfield, Ill.).

The kit can include one or more containers for the composition containing a VLA-4 antagonist described herein. In some embodiments, the kit contains separate containers, dividers or compartments for the composition and informational material. For example, the composition can be contained in a bottle, vial, IV admixture bag, IV infusion set, piggyback set or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. The containers of the kits can be air tight, waterproof (e.g., impermeable to changes in moisture or evaporation), and/or light-tight.

The invention is further illustrated by the following examples, which should not be construed as further limiting.

### EXAMPLES

### Example

### Effect of Natalizumab on Infarct Volume in Acute Ischemic Stroke

This example describes the determination of the effect of natalizumab on infarct volume in human subjects with acute ischemic stroke. The study is expected to demonstrate that one 300 mg dose of intravenous (IV) natalizumab reduces change in infarct volume from Baseline to Day 5 on magnetic resonance imaging (MRI) in subjects with acute ischemic stroke when given at ≦6 hours or at >6 to ≦9 hours from when they were last known normal (LKN). Additional study parameters, including the efficacy of natalizumab on change in infarct volume from Baseline to Day 30; efficacy of natalizumab on change in infarct volume from 24 hours to Day 5 and Day 30; the efficacy of natalizumab on clinical measures of stroke outcome; and the safety of natalizumab in subjects with acute ischemic stroke are expected to yield results that support the safety and efficacy of natalizumab in treatment of subjects having ischemic stroke. Further study parameters, including the effects of natalizumab on edema and intracranial hemorrhage (ICH); the efficacy of natalizumab on prevention of new or recurrent ischemic events; the efficacy of natalizumab on cognitive function; the effects of natalizumab on peripheral markers of inflammation, primarily α4 integrin expression on leukocyte subsets and other markers of inflammation that are thought to be upregulated in stroke; and the efficacy of natalizumab on functional outcomes of stroke are also expected to yield results that support the safety and efficacy of natalizumab in treatment of subjects having ischemic stroke.

### Study Design:

This is a Phase 2 multicenter, double-blind, placebo-controlled, randomized, parallel-group study of natalizumab in subjects with acute ischemic stroke, administered at ≦6 hours or at >6 to ≦9 hours from when the subjects were LKN. This study is expected to demonstrate the efficacy and safety of natalizumab over a 90-day period. The effect of natalizumab on infarct volume in acute ischemic stroke fulfilling the criteria for study entry, eligible subjects in both time windows will be randomized in a 1:1 ratio to receive a single dose of 300 mg IV natalizumab or placebo. Post-treatment assessments will be performed at the following time points after the start of study treatment administration: 12±3 hours, 24±6 hours, and Days 5 (or prior to discharge), 30, and 90.

This study will be conducted in subjects between the ages of 18 and 85 (inclusive) who have presented to a hospital with signs of acute ischemic stroke at ≦6 hours or at >6 to ≦9 hours from when they were LKN, an NIHSS score of ≧6 points, and at least 1 acute infarct with largest diameter of more than 2 cm on Baseline brain DWI.

Subjects will receive 1 dose of natalizumab (an IV infusion that will last for approximately 1 hour) that is initiated at ≦6 hours or at >6 to ≦9 hours from LKN. Post-treatment assessments will be performed at 12±3 hours, 24±6 hours, and Days 5 (or prior to discharge), 30, and 90 after the start of study treatment administration.

Subjects will be evaluated by one or more of the following: Brain MRI; NIHSS; mRS; Barthel Index; MoCA and SIS-16; serum concentrations of natalizumab at selected times after dosing; blood biomarkers of natalizumab target engagement; Lymphocyte subsets, including but not limited to, T cell, B cell, and natural killer cell analysis; Serum cytokines and other inflammatory markers of stroke; physical and neurological examinations; vital sign measurements: temperature, pulse rate, systolic and diastolic blood pressure, and respiratory rate; laboratory assessments: hematology and blood chemistry; anti-natalizumab antibodies; anti-JC virus antibodies; monitoring of AEs and SAEs; monitoring of concomitant medications and concomitant procedures.

### Endpoints and Statistical Analysis

The primary endpoint of the study will be a change in infarct volume from Baseline (diffusion-weighted imaging [DWI]) to Day 5 (fluid-attenuated inversion recovery [FLAIR]). The primary endpoint is the change in infarct volume from Baseline (DWI) to Day 5 (FLAIR). The primary analysis will be based on geometric mean (calculated as exponential of mean log relative growth). The geometric mean will be presented by treatment group and analyzed by analysis of covariance (ANCOVA) adjusting for Baseline DWI volume, Baseline perfusion, and treatment time window. The secondary analysis will be based on relative growth (calculated as Day 5 FLAIR divided by Baseline DWI) and absolute growth (calculated as Day 5 FLAIR minus Baseline DWI), and analyzed by non-parametric Wilcoxon's rank-sum test.

The secondary endpoint of the study will be a change in infarct volume from Baseline (DWI) to Day 30 (FLAIR); change in infarct volume from 24 hours (DWI) to Day 5 and Day 30 (FLAIR); change in National Institute of Health Stroke Scale (NIHSS) score from Baseline to 24 hours, Day 5, Day 30, and Day 90; Modified Rankin Scale (mRS) distribution at Day 5, Day 30, and Day 90; Barthel Index at Day 5, Day 30, and Day 90; and/or incidence of adverse events (AE) and serious; AEs (SAE). Further endpoints of the study may include volume of edema at 24 hours and Day 5; incidence of ICH, symptomatic or asymptomatic, at 24 hours, Day 5, and Day 30; incidence and volume of new infarcts at 24 hours, Day 5, and Day 30; score on Montreal Cognitive Assessment (MoCA) at Day 5, Day 30, and Day 90; evaluation of natalizumab saturation of α4 integrin expressed on leukocyte subsets and assessment of the effect of natalizumab treatment on other peripheral and stroke-related markers of inflammation, which may include, but is not limited to, interleukin (IL)-6, high sensitivity C-reactive protein, matrix metalloproteinase (MMP)-2, MMP-9, soluble intercellular adhesion molecule-1, soluble vascular cell adhesion molecule-1, IL-8, IL-10, and tumor necrosis factor alpha; and/or Stroke Impact Scale-16 (SIS-16) at Day 5, Day 30, and Day 90.

For the secondary endpoints, continuous variables will be summarized using summary statistics (mean, standard deviation, median, minimum, and maximum) by treatment group, and categorical variables will be presented using frequency distributions by time point and treatment group. Change in infarct volume based on geometric means will be analyzed by ANCOVA, adjusting for Baseline DWI volume, Baseline perfusion, and treatment time window. Change in infarct volume based on relative growth and absolute growth will be analyzed by non-parametric Wilcoxon's rank-sum test. Barthel Index will be analyzed by non-parametric Wilcoxon's rank-sum test, and by analysis of variance (ANOVA) or ANCOVA adjusting for Baseline DWI volume, Baseline perfusion, and treatment time window. Change in NIHSS from Baseline will be analyzed by ANOVA or ANCOVA adjusting for Baseline DWI volume, Baseline perfusion, and treatment time window. The mRS distribution will be analyzed by Van Elteren's test, unadjusted and adjusted for Baseline DWI volume, Baseline perfusion, and treatment time window. Univariate and multivariable regression analysis will be performed to assess the relationship between change in infarct volume and clinical outcome (NIHSS, mRS, and Barthel Index). Subgroup analysis by treatment time window and by Baseline DWI infarct size (<4 cm versus ≧4 cm, largest diameter) will be conducted for selected efficacy endpoints.

### References

Becker K, Kindrick D, Relton J, Harlan J, Winn R. Antibody to the α4 integrin decreases infarct size in transient focal cerebral ischemia in rats. Stroke 2001; 32: 206-211.

Gelderblom M, Leypoldt F, Steinbach K et al. Temporal and Spatial Dynamics of Cerebral Immune Cell Accumulation in Stroke. Stroke 2009; 40:1849-1857.

Liesz A, Zhou W, Mracsko E, et al. Inhibition of lymphocyte trafficking shields the brain against deleterious neuroinflammation after stroke. Brain 2011; 134:704-720.

Relton J K, Sloan K E, Frew E M, Whalley E T, Adams S P, Lobb R R. Inhibition of α4 integrin protects against transient focal cerebral ischemia in normotensive and hypertensive rats. Stroke 2001; 32:199-205.

Other embodiments are in the claims.

1. A method of treating a human subject having a stroke comprising: administering a VLA-4 antagonist to the subject within 12 hours or less after the onset of the stroke in the subject.

2. The method of claim 1, wherein the VLA-4 antagonist is administered within 9 hours or less after the onset of the stroke.

3. The method of claim 1, wherein the VLA-4 antagonist is administered within 6 hours or less after the onset of the stroke.

4. A method of treating a subject having a stroke comprising: administering a VLA-4 antagonist to the subject within more than 2 hours to 12 hours after the onset of stroke in the subject.

5. The method of claim 4, wherein the VLA-4 antagonist is administered within more than 2 to 9 hours or less after the onset of the stroke.

6. The method of claim 4, wherein the VLA-4 antagonist is administered within more than 2 hours to 6 hours or less after the onset of the stroke.

7. The method of claim 1, wherein the VLA-4 antagonist is an anti-VLA-4 antibody molecule.

8. The method of claim 7, wherein the anti-VLA-4 antibody molecule is a monoclonal, a humanized, a human, or a chimeric anti-VLA-4 antibody molecule.

9. The method of claim 7, wherein the VLA-4 antagonist is an α4-binding fragment of an anti-VLA-4 antibody.

10. The method of claim 9, wherein the α4 binding fragment is an Fab, Fab′, F(ab′)2, or Fv fragment.

11. The method of claim 7, wherein the anti-VLA-4 antibody molecule comprises one or more of HC CDR1, HC CDR2, HC CDR3, LC CDR1, LC CDR2 and LC CDR3 of natalizumab.

12. The method of claim 1, wherein the VLA-4 antagonist is natalizumab.

13. The method of claim 12, wherein natalizumab is administered at a dose of 200 mg to 400 mg.

14. The method of claim 1, wherein the stroke is a grade 4 stroke or higher as defined by the National Institute of Health Stroke Scale (NIHSS).

15. The method of claim 1, wherein the stroke is a grade 6 stroke or lower as defined by the National Institute of Health Stroke Scale (NIHSS).

16. The method of claim 1, wherein the stroke is a moderate stroke, a moderate to severe stroke or a severe stroke.

17. The method of claim 1, wherein the stroke is embolism-, thrombus- or hypoperfusion-associated stroke.

18. The method of claim 1, wherein the subject having the stroke does not have an intracranial hemorrhage.

19. The method of claim 12, wherein the subject has not received a previous treatment with a VLA-4 antagonist.

20. The method of claim 12, wherein the subject does not have or is not at risk for developing progressive multifocal leukoencephalopathy (PML).

21. The method of claim 1, wherein the VLA-4 antagonist is administered in combination with an additional agent or procedure.

22. The method of claim 21, wherein the VLA-4 antagonist is administered simultaneously with an additional agent or procedure.

23. The method of claim 21, wherein the VLA-4 antagonist is administered sequentially with an additional agent or procedure.

24. The method of claim 23, wherein the VLA-4 antagonist is administered after the additional agent or procedure.

25. The method of claim 23, wherein the VLA-4 antagonist is administered before the additional agent or procedure.

26. The method of claim 21, wherein the additional agent ameliorates one or more side effects associated with the administration of the VLA-4 antagonist.

27. The method of claim 26, wherein the additional agent can be one or more of a corticosteroid, an antihistamine, an H1 antagonist and an H2 antagonist.

28. The method of claim 21, wherein the additional agent is an agent which reduces one or more symptoms of stroke.

29. A method of treating a human subject having an ischemic stroke comprising: administering natalizumab to the subject within 9 hours or less after the onset of the stroke in the subject.

30. A method of treating a subject having an ischemic stroke comprising: administering natalizumab to the subject within more than 2 hours to 9 hours or less after the onset of the stroke in the subject.

31. The method of claim 29, wherein the natalizumab is administered at a dose of 300 mg.

32. The method of claim 29, wherein the stroke is an acute ischemic stroke of a grade 4 stroke or higher as defined by the National Institute of Health Stroke Scale (NIHSS).

33. The method of claim 29, wherein the stroke is a grade 6 stroke or lower as defined by the National Institute of Health Stroke Scale (NIHSS).

34. The method of claim 29, wherein the stroke is a moderate stroke, a moderate to severe stroke or a severe stroke.

35. The method of claim 29, wherein the stroke is an embolism-, thrombus- or hypoperfusion-associated stroke.

36. The method of claim 29, wherein the subject has not received a previous treatment with natalizumab.

37. The method of claim 29, wherein the subject does not have or is not at risk for developing progressive multifocal leukoencephalopathy (PML).

38. A method of treating a subject having an acute ischemic stroke of a grade 4 stroke or higher as defined by the National Institute of Health Stroke Scale (NIHSS), comprising: administering a VLA-4 antagonist to the subject after the onset of the stroke in the subject.

39. The method of claim 38, wherein the VLA-4 antagonist is an anti-VLA-4 antibody molecule.

40. The method of claim 38, wherein the VLA-4 antagonist is administered at a dose of 200 mg to 400 mg.
